SEQUENCE LISTING (36 sequences)

SEQ ID NO: 1 - protein, 20 aa, Artificial Sequence
  Description of Artificial Sequence Synthetic peptide
XXXXXXFSRTQWYXXXXXXX

SEQ ID NO: 2 - protein, 6 aa, Artificial Sequence
  Description of Artificial Sequence Synthetic peptide
IRVRXX

SEQ ID NO: 3 - protein, 7 aa, Artificial Sequence
  Description of Artificial Sequence Synthetic peptide
FSRTQWY

SEQ ID NO: 4 - protein, 20 aa, Artificial Sequence
  Description of Artificial Sequence Synthetic peptide
IRVRRLFSRTQWYLRIDRRK

SEQ ID NO: 5 - protein, 19 aa, Artificial Sequence
  Description of Artificial Sequence Synthetic peptide
RVRRLFSRTQWYLRIDRRK

SEQ ID NO: 6 - protein, 18 aa, Artificial Sequence
  Description of Artificial Sequence Synthetic peptide
VRRLFSRTQWYLRIDRRK

SEQ ID NO: 7 - protein, 17 aa, Artificial Sequence
  Description of Artificial Sequence Synthetic peptide
RRLFSRTQWYLRIDRRK

SEQ ID NO: 8 - protein, 16 aa, Artificial Sequence
  Description of Artificial Sequence Synthetic peptide
RLFSRTQWYLRIDRRK

SEQ ID NO: 9 - protein, 19 aa, Artificial Sequence
  Description of Artificial Sequence Synthetic peptide
IRVRRLFSRTQWYLRIDRK

SEQ ID NO: 10 - protein, 18 aa, Artificial Sequence
  Description of Artificial Sequence Synthetic peptide
RVRRLFSRTQWYLRIDRK

SEQ ID NO: 11 - protein, 17 aa, Artificial Sequence
  Description of Artificial Sequence Synthetic peptide
VRRLFSRTQWYLRIDRK

SEQ ID NO: 12 - protein, 16 aa, Artificial Sequence
  Description of Artificial Sequence Synthetic peptide
RRLFSRTQWYLRIDRK

SEQ ID NO: 13 - protein, 15 aa, Artificial Sequence
  Description of Artificial Sequence Synthetic peptide
RLFSRTQWYLRIDRK

SEQ ID NO: 14 - protein, 18 aa, Artificial Sequence
  Description of Artificial Sequence Synthetic peptide
IRVRRLFSRTQWYLRIDK

SEQ ID NO: 15 - protein, 17 aa, Artificial Sequence
  Description of Artificial Sequence Synthetic peptide
RVRRLFSRTQWYLRIDK

SEQ ID NO: 16 - protein, 16 aa, Artificial Sequence
  Description of Artificial Sequence Synthetic peptide
VRRLFSRTQWYLRIDK

SEQ ID NO: 17 - protein, 15 aa, Artificial Sequence
  Description of Artificial Sequence Synthetic peptide
RRLFSRTQWYLRIDK

SEQ ID NO: 18 - protein, 14 aa, Artificial Sequence
  Description of Artificial Sequence Synthetic peptide
RLFSRTQWYLRIDK

SEQ ID NO: 19 - protein, 19 aa, Artificial Sequence
  Description of Artificial Sequence Synthetic peptide
IRVRRCFSRTQWYCRIDRK

SEQ ID NO: 20 - protein, 18 aa, Artificial Sequence
  Description of Artificial Sequence Synthetic peptide
RVRRCFSRTQWYCRIDRK

SEQ ID NO: 21 - protein, 17 aa, Artificial Sequence
  Description of Artificial Sequence Synthetic peptide
VRRCFSRTQWYCRIDRK

SEQ ID NO: 22 - protein, 19 aa, Artificial Sequence
  Description of Artificial Sequence Synthetic peptide
IRVRCLFSRTQWYCRIDRK

SEQ ID NO: 23 - protein, 18 aa, Artificial Sequence
  Description of Artificial Sequence Synthetic peptide
RVRCLFSRTQWYCRIDRK

SEQ ID NO: 24 - protein, 18 aa, Artificial Sequence
  Description of Artificial Sequence Synthetic peptide
RVRCLFSRTQWYCRIDQK

SEQ ID NO: 25 - protein, 19 aa, Artificial Sequence
  Description of Artificial Sequence Synthetic peptide
IRVRRCFSRTQWYLCIDRK

SEQ ID NO: 26 - protein, 18 aa, Artificial Sequence
  Description of Artificial Sequence Synthetic peptide
RVRRCFSRTQWYLCIDRK

SEQ ID NO: 27 - protein, 17 aa, Artificial Sequence
  Description of Artificial Sequence Synthetic peptide
VRRCFSRTQWYLCIDRK

SEQ ID NO: 28 - protein, 19 aa, Artificial Sequence
  Description of Artificial Sequence Synthetic peptide
IRVRCLFSRTQWYLCIDRK

SEQ ID NO: 29 - protein, 18 aa, Artificial Sequence
  Description of Artificial Sequence Synthetic peptide
RVRCLFSRTQWYLCIDRK

SEQ ID NO: 30 - protein, 17 aa, Artificial Sequence
  Description of Artificial Sequence Synthetic peptide
VRCLFSRTQWYLCIDRK

SEQ ID NO: 31 - protein, 8 aa, Artificial Sequence
  Description of Artificial Sequence Synthetic peptide
IRVRXXFS

SEQ ID NO: 32 - protein, 7 aa, Artificial Sequence
  Description of Artificial Sequence Synthetic peptide
RVRXXFS

SEQ ID NO: 33 - protein, 10 aa, Artificial Sequence
  Description of Artificial Sequence Synthetic peptide
QWYXXIDXXK

SEQ ID NO: 34 - protein, 9 aa, Artificial Sequence
  Description of Artificial Sequence Synthetic peptide
QWYXXIDXX

SEQ ID NO: 35 - protein, 8 aa, Artificial Sequence
  Description of Artificial Sequence Synthetic peptide
QWYXXIDX

SEQ ID NO: 36 - protein, 6 aa, Artificial Sequence
  Description of Artificial Sequence Synthetic peptide
VRXXFS

(19) United States
(12) Patent Application Publication
(10) Pub. No.: US 2009/0042802 A1
(43) Pub. Date: Feb. 12, 2009

(54) COMPOUNDS AND PEPTIDES THAT BIND THE KGF RECEPTOR

(76) Inventors: YIJUN PAN, Union City, CA (US); Christopher Holmes, Saratoga, CA (US); Yvonne Angell, San Carlos, CA (US)

Correspondence Address: Goodwin Procter LLP;Attn: Patent Administrator, 135 Commonwealth Drive, Menlo Park, CA, 94025-1105, US

(21) Appl. No.: 12/143,684

(22) Filed: Jun. 20, 2008

Related U.S. Application Data
(60) Provisional application No. 60/945,720, filed on Jun. 22, 2007.

(51) Int. Cl. A61K 38/08; A61K 38/10; C07K 7/06; C07K 7/08; C07K 7/64

(52) U.S. Cl. 514/13; 530/326; 530/327; 530/328; 530/317; 514/14; 514/15; 514/16

(57) ABSTRACT

The present invention relates to peptide compounds that bind the KGF receptor. The invention also relates to therapeutic methods using such peptide compounds to treat disorders associated with defective or insufficient epithelial cell proliferation. Pharmaceutical compositions, which comprise the peptide compounds of the invention, and dosages are also provided.

### CROSS-REFERENCE TO RELATED APPLICATIONS

This application is a U.S. Non-Provisional Application claiming priority under 35 U.S.C. § 119(e) to U.S. Provisional Application Ser. No. 60/945,720, filed on Jun. 22, 2007, which is incorporated by reference in its entirety.

### FIELD OF THE INVENTION

The present invention provides, inter alia, peptides and compounds that bind and activate the FGF-R2(IIIb) receptor, i.e. the KGF receptor, or otherwise act as a FGF-R2(IIIb) agonist. The invention has application in the fields of biochemistry and medicinal chemistry and particularly provides FGF-R2(IIIb) binding peptides for use in the treatment of human diseases and conditions.

### BACKGROUND OF THE INVENTION

The use of certain peptides to promote wound healing has been attempted. For example, peptides believed to mimic fibroblast growth factor (FGF) (Safell U.S. Pat. No. 7,304,129 incorporated herein by reference in its entirety) and truncated forms of IL-10 (Ferguson et al. U.S. Published Application 20080139478 and Ferguson et al. U.S. Pat. No. 7,052,684, each of which is incorporated herein by reference in its entirety) have been reported as having potential to promote wound healing. The fibroblast growth factor receptors (FGFRs) are cell surface tyrosine kinase receptors that are implicated in numerous processes during cell growth and development. KGF (keratinocyte growth factor) or FGF-7 (LaRochelle et al., Biochemistry, 38, 1765 (1999); Osslund., et al., Protein Science, 7, 1681-1690 (1998); Ye., et al., Biochemistry, 40, 14429-14439 (2001); Bottaro, et al., J. Biol. Chem. 268, 9180-9183 (1993); Pellegrini et al., Nature, 2000, 407, 102 (2000)) is a member of the FGF family that binds the FGF receptor splice variant: FGF-R2(IIIb), i.e., KGF-R. KGF mediates proliferation of epithelial cells and has potential for treatment of oral mucositis, venous ulcers and ulcerative colitis.

### SUMMARY OF THE INVENTION

In one aspect, the present invention provides peptides that bind to the KGF receptor. In one embodiment, the peptide includes an amino acid sequence Xaa_{b}-Phe-Ser-Arg-Thr-Gln-Trp-Tyr-Xaa_{c} (SEQ ID NO: 1) that binds KGF receptor, wherein Xaa_{b} is between 0 and 6 amino acids and Xaa_{c} is between 0 and 7 amino acids. In other embodiments, Xaa_{b} is Ile-Arg-Val-Arg-Xaa₁-Xaa₂ (SEQ ID NO:2); Xaa_{b} is Arg-Val-Arg-Xaa₁-Xaa₂; Xaa_{b} is Val-Arg-Xaa₁-Xaa₂, or Xaa_{b} is Arg-Xaa₁-Xaa₂, wherein Xaa₁ is Arg or Cys and Xaa₂ is Leu or Cys. In another embodiment, Xaa_{c} is Xaa₁-Xaa₂-Ile-Asp-Xaa₃-Xaa₄-Lys; Xaa_{c} is Xaa₂₁-Xaa₂-Ile-Asp-Xaa₃-Xaa₄; or Xaa_{c} is Xaa₁-Xaa₂-Ile-Asp-Xaa₃, wherein Xaa₁ is Leu or Cys; Xaa₂ is Arg or Cys; and Xaa₃ is Arg, Lys, or Gln; and Xaa₄ is Arg or Lys.

In all embodiments, the amino acid sequence is Phe-Ser-Arg-Thr-Gln-Trp-Tyr (SEQ ID NO:3).

In another embodiment, the peptide includes an amino acid sequence Xaa_{b}-Arg-Thr-Gln-Xaa_{c} that binds the KGF receptor, wherein Xaa_{b} is between 2 and 8 amino acids and Xaa_{b} is between 2 and 9 amino acids. In other embodiments, Xaa_{b} is Ile-Arg-Val-Arg-Xaa₁-Xaa₂-Phe-Ser (SEQ ID NO:31); Arg-Val-Arg-Xaa₁-Xaa₂-Phe-Ser (SEQ ID NO:32); Val-Arg-Xaa₁-Xaa₂-Phe-Ser, or Arg-Xaa₁-Xaa₂-Phe-Ser, wherein Xaa₁ is Arg or Cys and Xaa₂ is Leu or Cys. In another embodiment, Xaa_{c} is Gln-Trp-Tyr-Xaa₁-Xaa₂-Ile-Asp-Xaa₃-Xaa₄-Lys (SEQ ID NO:33); Gln-Trp-Tyr-Xaa₁-Xaa₂-Ile-Asp-Xaa₃-Xaa₄ (SEQ ID NO:34); or Gln-Trp-Tyr-Xaa₁-Xaa₂-Ile-Asp-Xaa₃ (SEQ ID NO:35), wherein Xaa₁ is Leu or Cys; Xaa₂ is Arg or Cys; and Xaa₃ is Arg, Lys, or Gln; and Xaa₄ is Arg or Lys.

In all embodiments, the N-terminus of the peptide is acetylated. In all embodiments, the amino acid sequence is a monomer, a dimer, or a homodimer. In all embodiments, the amino acid sequence is cyclized. In one embodiment, the amino acid sequence is cyclized by an intramolecular disulfide bond formed between two cysteine residues.

In another aspect, the present invention provides peptide dimers. In one embodiment, the peptide dimer contains (a) a first peptide chain; (b) a second peptide chain; and (c) a linking moiety connecting the first and the second peptide chains, wherein at least one of the peptide chains includes an amino acid sequence Xaa_{b}-Phe-Ser-Arg-Thr-Gln-Trp-Tyr-Xaa_{c} (SEQ ID NO: 1) that binds KGF receptor, wherein Xaa_{b} is between 0 and 6 amino acids and Xaa_{c} is between 0 and 7 amino acids.

In another embodiment, the first and/or the second peptide chain of the peptide dimer contain an amino acid sequence Phe-Ser-Arg-Thr-Gln-Trp-Tyr (SEQ ID NO:3). In other embodiments, the linking moiety is a lysine residue.

In one other aspect, the present invention provides pharmaceutical compositions. In one embodiment, the pharmaceutical composition contains (i) a peptide that includes an amino acid sequence Xaa_{b}-Phe-Ser-Arg-Thr-Gln-Trp-Tyr-Xaa_{c}(SEQ ID NO:1) that binds KGF receptor, wherein Xaa_{b} is between 0 and 6 amino acids and Xaa_{c} is between 0 and 7 amino acids; and (ii) a pharmaceutically acceptable carrier. In another embodiment, the pharmaceutical composition contains (i) a peptide dimer including (a) a first peptide chain; (b) a second peptide chain; and (c) a linking moiety connecting the first and the second peptide chains, wherein at least one of the peptide chains includes an amino acid sequence Xaa_{b}-Phe-Ser-Arg-Thr-Gln-Trp-Tyr-Xaa_{c} (SEQ ID NO:1) that binds KGF receptor, wherein Xaa_{b} is between 0 and 6 amino acids and Xaa is between 0 and 7 amino acids; and (ii) a pharmaceutically acceptable carrier. In other embodiments, the pharmaceutical composition contains a peptide with the amino acid sequence Phe-Ser-Arg-Thr-Gln-Trp-Tyr (SEQ ID NO:3).

In another aspect, the present invention provides methods of treatment. In one embodiment, the present invention provides a method of treating a subject, including the step of administering to a subject having a disorder characterized by a need for epithelial cell proliferation a therapeutically effective amount of a peptide that includes an amino acid sequence Xaa_{b}-Phe-Ser-Arg-Thr-Gln-Trp-Tyr-Xaa_{c} (SEQ ID NO:1) that binds KGF receptor, wherein Xaa_{b} is between 0 and 6 amino acids and Xaa_{c} is between 0 and 7 amino acids. In another embodiment, the method includes the step of administering a therapeutically effective amount of a peptide dimer that includes an amino acid sequence Xaa_{b}-Phe-Ser-Arg-Thr-Gln-Trp-Tyr-Xaa_{c}(SEQ ID NO:1) that binds KGF receptor, wherein Xaa_{b} is between 0 and 6 amino acids and Xaa_{c} is between 0 and 7 amino acids. In other embodiments, the disorders suitable for treatment by the methods of the present invention include those for which the stimulation of epithelial cell proliferation is desirable. The disorder may be characterized by insufficient or defective epithelial cell proliferation. For example, suitable disorders include, without limitation, various types of wounds such as, for example, wounds caused by trauma, burns (e.g. thermal, chemical, and/or radiation burns), surgery, and radiation damage (e.g. from radiotherapy). In another embodiment, the disorders include oral mucositis, venous ulcers, diabetic ulcers, decubitus ulcers (bed sores), and ulcerative colitis.

In all embodiments, the subject is a mammal, preferably a human subject.

In one aspect, the present invention provides uses of a peptide or peptide dimer for the manufacture of medicaments for the promotion of wound healing. In one embodiment, the present invention provides a use of a peptide or a peptide dimer described herein in the manufacture of a medicament to treat a subject having a disorder characterized by a need for epithelial cell proliferation.

In one aspect, the peptides and compounds bind and activate the KGF-R or FGF-R2(IIIb) receptor or otherwise act as a KGF-R or FGF-R2(IIIb) agonist. In one embodiment, the present invention provides a compound comprising a peptide that binds to KGF-R or FGF-R2(IIIb) and comprises a sequence of amino acids Phe-Ser-Arg-Thr-Gln-Trp-Tyr (SEQ ID NO:3). In another embodiment, the peptide is approximately 14 to 20 amino acids in length. In other embodiments, the peptide is selected from a peptide listed in Table 1. In some embodiments, the compound comprises a peptide that is a monomer, a peptide that is a dimer, or a peptide that is a homodimer. Peptides may be dimerized via a lysine residue at the C-terminus using a bi-functional linker. In addition, the compounds or peptides may contain cysteine residues for the purpose of introducing an intramolecular disulfide bridge or constraint at various locations in the amino acid sequence. In one embodiment, the disulfide constraint will be of varying loop sizes as illustrated in by the amino acids shown as SEQ ID NOS:19-30 in Table 1.

In one embodiment, the present invention provides a compound comprising a peptide homo-dimer that binds to the FGF-R2(IIIb) receptor and comprises a sequence of amino acids Phe-Ser-Arg-Thr-Gln-Trp-Tyr (SEQ ID NO:3) where each amino acid is indicated by standard one letter abbreviation. In all embodiments, the peptides or compounds may contain an intramolecular disulfide constraint as described herein. In another aspect, the present invention provides medicaments and methods of using the same for the treatment of conditions in a subject that relate to epithelial cell proliferation. In one embodiment, the condition suitable for treatment includes those conditions that would benefit from wound healing as a result of epithelial cell proliferation. In one embodiment, the condition is associated with a side effects of chemotherapy. Suitable conditions include, without limitation, oral mucositis, venous ulcers, and ulcerative colitis. In another embodiment, the invention provides methods of treating the conditions described herein comprising administering the compounds or peptides described herein in a pharmaceutically acceptable form. In all embodiments, the subject is a mammalian subject, preferably human. In one other aspect, the present invention provides methods of making the peptides or compounds described herein.

### DETAILED DESCRIPTION OF THE INVENTION

The present invention relates to peptides and compounds that bind to an FGF receptor or otherwise act as an FGF receptor agonist, as well as methods of treating human diseases or conditions using the same. In addition, methods of synthesizing the peptides and compounds described herein are provided by the present invention.

### DEFINITIONS

As used herein, the terms “KGF Receptor”; “KGF-R”; and “FGF-R2(IIIb)” refer to the FGF receptor splice variant FGF-R2(IIIb), also known in the art as the KGF receptor.

As used herein, the term “polypeptide” or “protein” refers to a polymer of amino acid monomers that are alpha amino acids joined together through amide bonds. Polypeptides are therefore at least two amino acid residues in length, and are usually longer. Generally, the term “peptide” refers to a polypeptide that is only a few amino acid residues in length.

As used herein, the phrase “pharmaceutically acceptable” refers to molecular entities and compositions that are “generally regarded as safe,” e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

As used herein the term “agonist” refers to a biologically active ligand which binds to its complementary biologically active receptor and activates the latter either to cause a biological response in the receptor, or to enhance preexisting biological activity of the receptor.

### Peptides and Peptide Dimers

The present invention relates to peptides that compete with KGF for binding of the KGF-R, showing potential for a potent agonist of KGF-R. The peptides of the present invention preferably inhibit the binding of KGF and KGF-R with a potency characterized by an IC₅₀ concentration. In one embodiment, the IC₅₀ concentration for a peptide monomer is about 10-100 nM, about 20-90 nM, about 30-80 nM, about 40-70 nM, or about 50-60 nM. In one preferred embodiment, the IC₅₀ concentration is about 15-97 nM. In another embodiment, the IC₅₀ concentration for a peptide dimer is less than about 100 nM, about 90 nM, about 80 nM, about 70 nM, about 60 nM, about 50 nM, about 40 nM, about 30 nM, about 20 nM, or about 10 nM. In one other embodiment, the IC₅₀ concentration for a peptide monomer is about 100 nM, about 90 nM, about 80 nM, about 70 nM, about 60 nM, about 50 nM, about 40 nM, about 30 nM, about 20 nM, or about 10 nM.

In one embodiment, the IC₅₀ concentration for a peptide dimer about 1-10 nM, about 2-9 nM, about 4-8 nM, or about 5-7 nM. In one preferred embodiment, the IC₅₀ concentration is 4-8 nM. In another embodiment, the IC₅₀ concentration for a peptide dimer is less than about 10 nM, about 9 nM, about 8 nM, about 7 nM, about 6 nM, about 5 nM, about 4 nM, about 3 nM, about 2 nM, or about 1 nM.

In another embodiment, the IC₅₀ concentration for a peptide dimer is about 10 nM, about 9 nM, about 8 nM, about 7 nM, about 6 nM, about 5 nM, about 4 nM, about 3 nM, about 2 nM, or about 1 nM.

These peptides preferably are of about 7 to about 45 amino acids in length and more preferably of about 14 to about 20 amino acids in length. In other embodiments, the peptides of the present invention comprise an amino acid sequence of at least 3, 4, 5, 6, 7, 8, 9, 10, 11, 12, 13, 14, 15, 16, 17, 18, 19, 20, 21, 22, 23, 24, or 25 amino acids in length. A polypeptide, in contrast with a peptide, may comprise any number of amino acid residues. Hence, the term polypeptide included peptides as well as longer sequences of amino acids.

In one aspect, the present invention provides peptide monomers and peptide dimers that bind to the KGF receptor. In one embodiment, the peptide monomer or dimer includes an amino acid sequence Xaa_{b}-Phe-Ser-Arg-Thr-Gln-Trp-Tyr-Xaa_{c} (SEQ ID NO:1) that binds KGF receptor, wherein Xaa_{b} is between 0 and 6 amino acids and Xaa_{c} is between 0 and 7 amino acids. In other embodiments, Xaa_{b} is Ile-Arg-Val-Arg-Xaa₁-Xaa₂ (SEQ ID NO:2); Xaa_{b} is Arg-Val-Arg-Xaa₁-Xaa₂; Xaa_{b} is Val-Arg-Xaa₁-Xaa₂, or Xaa_{b} is Arg-Xaa₁-Xaa₂, wherein Xaa₁ is Arg or Cys and Xaa₂ is Leu or Cys. In another embodiment, Xaa_{c} is Xaa₁-Xaa₂-Ile-Asp-Xaa₃-Xaa₄-Lys; Xaa_{c} is Xaa₁-Xaa₂-Ile-Asp-Xaa₃-Xaa₄; or Xaa_{c} is Xaa₁-Xaa₂-Ile-Asp-Xaa₃, wherein Xaa₁ is Leu or Cys; Xaa₂ is Arg or Cys; and Xaa₃ is Arg, Lys, or Gln; and Xaa₄ is Arg or Lys. In one embodiment, the peptide monomer or dimer includes one or more amino acid sequences selected from Table 1.

Table 1 provides examples of the peptides of the present invention.

TABLE 1

| | |
|---|---|
| | SEQ ID |
| SEQUENCE | NO: |

| | |
|---|---|
| Ac-Ile-Arg-Val-Arg-Arg-Leu-Phe-Ser-Arg-Thr- | 4 |
| Gln-Trp-Tyr-Leu-Arg-Ile-Asp-Arg-Arg-Lys-NH₂ | |
| Ac-Arg-Val-Arg-Arg-Leu-Phe-Ser-Arg-Thr-Gln- | 5 |
| Trp-Tyr-Leu-Arg-Ile-Asp-Arg-Arg-Lys-NH₂ | |
| Ac-Val-Arg-Arg-Leu-Phe-Ser-Arg-Thr-Gln-Trp- | 6 |
| Tyr-Leu-Arg-Ile-Asp-Arg-Arg-Lys-NH₂ | |
| Ac-Arg-Arg-Leu-Phe-Ser-Arg-Thr-Gln-Trp-Tyr- | 7 |
| Leu-Arg-Ile-Asp-Arg-Arg-Lys-NH₂ | |
| Ac-Arg-Leu-Phe-Ser-Arg-Thr-Gln-Trp-Tyr-Leu- | 8 |
| Arg-Ile-Asp-Arg-Arg-Lys-NH₂ | |
| Ac-Ile-Arg-Val-Arg-Arg-Leu-Phe-Ser-Arg-Thr- | 9 |
| Gln-Trp-Tyr-Leu-Arg-Ile-Asp-Arg-Lys-NH₂ | |
| Ac-Arg-Val-Arg-Arg-Leu-Phe-Ser-Arg-Thr-Gln- | 10 |
| Trp-Tyr-Leu-Arg-Ile-Asp-Arg-Lys-NH₂ | |
| Ac-Val-Arg-Arg-Leu-Phe-Ser-Arg-Thr-Gln-Trp- | 11 |
| Tyr-Leu-Arg-Ile-Asp-Arg-Lys-NH₂ | |
| Ac-Arg-Arg-Leu-Phe-Ser-Arg-Thr-Gln-Trp-Tyr- | 12 |
| Leu-Arg-Ile-Asp-Arg-Lys-NH₂ | |
| Ac-Arg-Leu-Phe-Ser-Arg-Thr-Gln-Trp-Tyr-Leu- | 13 |
| Arg-Ile-Asp-Arg-Lys-NH₂ | |
| Ac-Ile-Arg-Val-Arg-Arg-Leu-Phe-Ser-Arg-Thr- | 14 |
| Gln-Trp-Tyr-Leu-Arg-Ile-Asp-Lys-NH₂ | |
| Ac-Arg-Val-Arg-Arg-Leu-Phe-Ser-Arg-Thr-Gln- | 15 |
| Trp-Tyr-Leu-Arg-Ile-Asp-Lys-NH₂ | |
| Ac-Val-Arg-Arg-Leu-Phe-Ser-Arg-Thr-Gln-Trp- | 16 |
| Tyr-Leu-Arg-Ile-Asp-Lys-NH₂ | |
| Ac-Arg-Arg-Leu-Phe-Ser-Arg-Thr-Gln-Trp-Tyr- | 17 |
| Leu-Arg-Ile-Asp-Lys-NH₂ | |
| Ac-Arg-Leu-Phe-Ser-Arg-Thr-Gln-Trp-Tyr-Leu- | 18 |
| Arg-Ile-Asp-Lys-NH₂ | |
| Ac-Ile-Arg-Val-Arg-Arg-Cys-Phe-Ser-Arg-Thr- | 19 |
| Gln-Trp-Tyr-Cys-Arg-Ile-Asp-Arg-Lys-NH₂ | |
| Ac-Arg-Val-Arg-Arg-Cys-Phe-Ser-Arg-Thr-Gln- | 20 |
| Trp-Tyr-Cys-Arg-Ile-Asp-Arg-Lys-NH₂ | |
| Ac-Val-Arg-Arg-Cys-Phe-Ser-Arg-Thr-Gln-Trp- | 21 |
| Tyr-Cys-Arg-Ile-Asp-Arg-Lys-NH₂ | |
| Ac-Ile-Arg-Val-Arg-Cys-Leu-Phe-Ser-Arg-Thr- | 22 |
| Gln-Trp-Tyr-Cys-Arg-Ile-Asp-Arg-Lys-NH₂ | |
| Ac-Arg-Val-Arg-Cys-Leu-Phe-Ser-Arg-Thr-Gln- | 23 |
| Trp-Tyr-Cys-Arg-Ile-Asp-Arg-Lys-NH₂ | |
| Ac-Arg-Val-Arg-Cys-Leu-Phe-Ser-Arg-Thr-Gln- | 24 |
| Trp-Tyr-Cys-Arg-Ile-Asp-Gln-Lys-NH₂ | |
| Ac-Ile-Arg-Val-Arg-Arg-Cys-Phe-Ser-Arg-Thr- | 25 |
| Gln-Trp-Tyr-Leu-Cys-Ile-Asp-Arg-Lys-NH₂ | |
| Ac-Arg-Val-Arg-Arg-Cys-Phe-Ser-Arg-Thr-Gln- | 26 |
| Trp-Tyr-Leu-Cys-Ile-Asp-Arg-Lys-NH₂ | |
| Ac-Val-Arg-Arg-Cys-Phe-Ser-Arg-Thr-Gln-Trp- | 27 |
| Tyr-Leu-Cys-Ile-Asp-Arg-Lys-NH₂ | |
| Ac-Ile-Arg-Val-Arg-Cys-Leu-Phe-Ser-Arg-Thr- | 28 |
| Gln-Trp-Tyr-Leu-Cys-Ile-Asp-Arg-Lys-NH₂ | |
| Ac-Arg-Val-Arg-Cys-Leu-Phe-Ser-Arg-Thr-Gln- | 29 |
| Trp-Tyr-Leu-Cys-Ile-Asp-Arg-Lys-NH₂ | |
| Ac-Val-Arg-Cys-Leu-Phe-Ser-Arg-Thr-Gln-Trp- | 30 |
| Tyr-Leu-Cys-Ile-Asp-Arg-Lys-NH₂ | |

In one embodiment, the present invention provides peptide monomers or peptide homodimers that contain an amino acid shown as SEQ ID NO:3-18 in Table 1. In one embodiment, the present invention provides peptide homodimers containing an amino acid shown as SEQ ID NO:3-18 in Table 1. In another embodiment, the present invention provides a peptide monomer or peptide dimer containing an amino acid sequence shown as SEQ ID NO:19-30 that includes a disulfide bond constraint between two Cys residues.

The peptides of this invention may be monomers, homo- or hetero-dimers, or other homo- or hetero-multimers. The term “homo” means comprising identical monomers; thus, for example, a homodimer of the present invention is a peptide comprising two identical monomers. The term “hetero” means comprising different monomers; thus, for example, a heterodimer of the present invention is a peptide comprising two non-identical monomers. The peptide multimers of the invention may be trimers, tetramers, pentamers, or other higher order structures. Moreover, such dimers and other multimers may be heterodimers or heteromultimers. The peptide monomers of the present invention may be degradation products (e.g., oxidation products of methionine or deamidated glutamine, arganine, and C-terminus amide). Such degradation products may be used in and are therefore considered part of the present invention. In preferred embodiments, the heteromultimers of the invention comprise multiple peptides that are all KGF-R binding peptides. In highly preferred embodiments, the multimers of the invention are homomultimers: i.e., they comprise multiple KGF-R binding peptides of the same amino acid sequence.

Accordingly, the present invention also relates to homo- or hetero-dimeric peptides that bind KGF-R, which show potential for potent agonistic activity. In preferred embodiments, the peptide dimers of the invention comprise two peptides that are both KGF-R agonist peptides. These peptide monomers preferably are of about 8 to about 45 amino acids in length and more preferably of about 14 to about 20 amino acids in length. In other embodiments, the peptides of the present invention comprise an amino acid sequence of at least 3, 4, 5, 6, 7, 8, 9, 10, 11, 12, 13, 14, 15, 16, 17, 18, 19, 20, 21, 22, 23, 24, or 25 amino acids in length.

These preferred peptide dimers agonists comprise two peptide monomers. In particularly preferred embodiments, the dimers of the invention comprise two KGF-R agonist peptides of the same amino acid sequence. Stereoisomers (e.g., D-amino acids) of the twenty conventional amino acids, unnatural amino acids such as a,a-disubstituted amino acids, N-alkyl amino acids, lactic acid, and other unconventional amino acids may also be suitable components for compounds of the present invention. Examples of unconventional amino acids include, but are not limited to: beta-alanine, 3-pyridylalanine, 4-hydroxyproline, O-phosphoserine, N-methylglycine, N-acetylserine, N-formylmethionine, 3-methylhistidine, 5-hydroxylysine, nor-leucine, and other similar amino acids and imino acids.

Other modifications are also possible, including modification of the amino terminus, modification of the carboxy terminus, replacement of one or more of the naturally occurring genetically encoded amino acids with an unconventional amino acid, modification of the side chain of one or more amino acid residues, peptide phosphorylation, and the like. A preferred amino terminal modification is acetylation (e.g., with acetic acid or a halogen substituted acetic acid). In preferred embodiments an N-terminal glycine is acetylated to N-acetylglycine (AcG). In preferred embodiments, a C-terminal glycine is N-methylglycine (MeG, also known as sarcosine).

In preferred embodiments, the peptide monomers or peptide dimers of the invention contain an intramolecular disulfide bond between the two cysteine residues of the core sequence.

The present invention also provides conjugates of these peptide monomers. Thus, according to a preferred embodiment, the monomeric peptides of the present invention are dimerized or oligomerized, thereby enhancing their KGF-R binding properties.

In one embodiment, the peptide monomers of the invention may be oligomerized using the biotin/streptavidin system. Biotinylated analogs of peptide monomers may be synthesized by standard techniques. For example, the peptide monomers may be C-terminally biotinylated. These biotinylated monomers are then oligomerized by incubation with streptavidin [e.g., at a 4:1 molar ratio at room temperature in phosphate buffered saline (PBS) or HEPES-buffered RPMI medium (Invitrogen) for 1 hour]. In a variation of this embodiment, biotinylated peptide monomers may be oligomerized by incubation with any one of a number of commercially available anti-biotin antibodies [e.g., goat anti-biotin IgG from Kirkegaard & Perry Laboratories, Inc. (Washington, D.C.)].

In preferred embodiments, the peptide monomers of the invention are dimerized by covalent attachment to at least one linker moiety. The linker (L_{K}) moiety is preferably, although not necessarily, a C₁₋₁₂ linking moiety optionally terminated with one or two—NH—linkages and optionally substituted at one or more available carbon atoms with a lower alkyl substituent. Preferably the linker L_{K} comprises—NH—R—NH—wherein R is a lower (C₁₋₆) alkylene substituted with a functional group such as a carboxyl group or an amino group that enables binding to another molecular moiety (e.g., as may be present on the surface of a solid support). Most preferably the linker is a lysine residue or a lysine amide (a lysine residue wherein the carboxyl group has been converted to an amide moiety—CONH₂). In preferred embodiments, the linker bridges the C-termini of two peptide monomers, by simultaneous attachment to the C-terminal amino acid of each monomer.

In an additional embodiment, polyethylene glycol (PEG) may serve as the linker L_{K} that dimerizes two peptide monomers: for example, a single PEG moiety may be simultaneously attached to the N-termini of both peptide chains of a peptide dimer.

In yet another additional embodiment, the linker (L_{K}) moiety is preferably, but not necessarily, a molecule containing two carboxylic acids and optionally substituted at one or more available atoms with an additional functional group such as an amine capable of being bound to one or more PEG molecules. Such a molecule can be depicted as: CO—(CH₂), —X—(CH₂), CO—where n is an integer from 0 to 10, m is an integer from 1 to 10, X is selected from O, S, N(CH₂)ₚNR₁, NCO(CH₂)ₚNR₁, and CHNR₁, R₁ is selected from H, Boc, Cbz, etc., and p is an integer from 1 to 10.

In preferred embodiments, one amino group of each of the peptides form an amide bond with the linker L_{K}. In particularly preferred embodiments, the amino group of the peptide bound to the linker L_{K} is the epsilon amine of a lysine residue or the alpha amine of the N-terminal residue, or an amino group of the optional spacer molecule.

Those of ordinary skill in the art will appreciate other linker strategies suitable for use with the peptides of the present invention (see U.S. Published Application 20080108564, hereby incorporated by reference in its entirety).

Generally, although not necessarily, peptide dimers will also contain one or more intramolecular disulfide bonds between cysteine residues of the peptide monomers. Preferably, the two monomers contain at least one intramolecular disulfide bond. Most preferably, both monomers of a peptide dimer contain an intramolecular disulfide bond, such that each monomer contains a cyclic group.

A peptide monomer or dimer may further comprise one or more spacer moieties. Such spacer moieties may be attached to a peptide monomer or to a peptide dimer. Preferably, such spacer moieties are attached to the linker L_{K} moiety that connects the monomers of a peptide dimer. For example, such spacer moieties may be attached to a peptide dimer via the carbonyl carbon of a lysine linker, or via the nitrogen atom of an iminodiacetic acid linker. For example, such a spacer may connect the linker of a peptide dimer to an attached water soluble polymer moiety or a protecting group. In another example, such a spacer may connect a peptide monomer to an attached water soluble polymer moiety.

In one embodiment, the spacer moiety is a C₁₋₁₂ linking moiety optionally terminated with—NH-linkages or carboxyl (—COOH) groups, and optionally substituted at one or more available carbon atoms with a lower alkyl substituent. In one embodiment, the spacer is R—COOH wherein R is a lower (C₁₋₆) alkylene optionally substituted with a functional group such as a carboxyl group or an amino group that enables binding to another molecular moiety. For example, the spacer may be a glycine (G) residue, or an amino hexanoic acid. In preferred embodiments the amino hexanoic acid is 6-amino hexanoic acid (Ahx). For example, where the spacer 6-amino hexanoic acid (Ahx) is bound to the N-terminus of a peptide, the peptide terminal amine group may be linked to the carboxyl group of Ahx via a standard amide coupling. In another example, where Ahx is bound to the C-terminus of a peptide, the amine of Ahx may be linked to the carboxyl group of the linker via a standard amide coupling.

In other embodiments, the spacer is —NH—R—NH—wherein R is a lower (C₁₋₆) alkylene substituted with a functional group such as a carboxyl group or an amino group that enables binding to another molecular moiety. For example, the spacer may be a lysine (K) residue or a lysine amide (K—NH₂, a lysine residue wherein the carboxyl group has been converted to an amide moiety—CONH₂).

The peptide monomers, dimers, or multimers of the invention may further comprise one or more water soluble polymer moieties. Preferably, these polymers are covalently attached to the peptide compounds of the invention. Preferably, for therapeutic use of the end-product preparation, the polymer will be pharmaceutically acceptable. One skilled in the art will be able to select the desired polymer based on such considerations as whether the polymer-peptide conjugate will be used therapeutically, and if so, the desired dosage, circulation time, resistance to proteolysis, and other considerations. The water soluble polymer may be, for example, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), poly(n-vinyl pyrrolidone)polyethylene glycol, propropylene glycol homopolymers, polypropylene oxide/ethylene oxide copolymers, and polyoxyethylated polyols. A preferred water soluble polymer is PEG.

The polymer may be of any molecular weight, and may be branched or unbranched. A preferred PEG for use in the present invention comprises linear, unbranched PEG having a molecular weight that is greater than 10 kilodaltons (kD) and is more preferably between about 20 and 60 kD in molecular weight. Still more preferably, the linear unbranched PEG moiety should have a molecular weight of between about 20 and 40 kD, with 20 kD PEG being particularly preferred. It is understood that in a given preparation of PEG, the molecular weights will typically vary among individual molecules. Some molecules will weight more, and some less, than the stated molecular weight. Such variation is generally reflect by use of the word “about” to describe molecular weights of the PEG molecules.

The number of polymer molecules attached may vary; for example, one, two, three, or more water soluble polymers may be attached to a KGF-R binding peptide of the invention. The multiple attached polymers may be the same or different chemical moieties (e.g., PEGs of different molecular weight). Thus, in a preferred embodiment the invention contemplates KGF-R binding peptides having two or more PEG moieties attached thereto. Preferably, both of the PEG moieties are linear, unbranched PEG each preferably having a molecular weight of between about 10 and about 60 kD. More preferably, each linear unbranched PEG moiety has a molecular weight that is between about 20 and 40 kD, and still more preferably between about 20 and 30 kD with a molecular weight of about 20 kD for each linear PEG moiety being particularly preferred. However, other molecular weights for PEG are also contemplated in such embodiments. For example, the invention contemplates and encompasses KGF-R binding peptides having two or more linear unbranched PEG moieties attached thereto, at least one or both of which has a molecular weight between about 20 and 40 kD or between about 20 and 30 kD. In other embodiments the invention contemplates and encompasses KGF-R binding peptides having two or more linear unbranched PEG moieties attached thereto, at least one of which has a molecular weight between about 40 and 60 kD.

In one embodiment, PEG may serve as a linker that dimerizes two peptide monomers. In one embodiment, PEG is attached to at least one terminus (N-terminus or C-terminus) of a peptide monomer or dimer. In another embodiment, PEG is attached to a spacer moiety of a peptide monomer or dimer. In a preferred embodiment PEG is attached to the linker moiety of a peptide dimer. In a highly preferred embodiment, PEG is attached to a spacer moiety, where said spacer moiety is attached to the linker L_{K} moiety that connects the monomers of a peptide dimer. In particularly preferred embodiments, PEG is attached to a spacer moiety, where said spacer moiety is attached to a peptide dimer via the carbonyl carbon of a lysine linker, or the amide nitrogen of a lysine amide linker.

Peptides and peptide sequences encompassed by the present invention, including peptide monomers and dimers, are shown Table 1, which describes individual peptides and peptide sequences by reference to Sequence Identification Numbers (SEQ ID NOs.).

The peptide sequences of the present invention can be present alone or in conjunction with N-terminal and/or C-terminal extensions of the peptide chain. Such extensions may be naturally encoded peptide sequences optionally with or substantially without non-naturally occurring sequences; the extensions may include any additions, deletions, point mutations, or other sequence modifications or combinations as desired by those skilled in the art. For example and not limitation, naturally-occurring sequences may be full-length or partial length and may include amino acid substitutions to provide a site for attachment of carbohydrate, PEG, other polymer, or the like via side chain conjugation. In a variation, the amino acid substitution results in humanization of a sequence to make in compatible with the human immune system. Fusion proteins of all types are provided, including immunoglobulin sequences adjacent to or in near proximity to the KGF-R activating sequences of the present invention with or without a non-immunoglobulin spacer sequence. One type of embodiment is an immunoglobulin chain having the KGF-R activating sequence in place of the variable (V) region of the heavy and/or light chain.

### Preparation of the Peptide Compounds of the Invention:

Peptide Synthesis—The peptides of the invention may be prepared by classical methods known in the art. These standard methods include exclusive solid phase synthesis, partial solid phase synthesis methods, fragment condensation, classical solution synthesis, and recombinant DNA technology [See, e.g., Merrifield J. Am. Chem. Soc. 1963 85:2149].

In one embodiment, the peptide monomers of a peptide dimer are synthesized individually and dimerized subsequent to synthesis. In preferred embodiments the peptide monomers of a dimer have the same amino acid sequence.

In particularly preferred embodiments, the peptide monomers of a dimer are linked via their C-termini by a linker L_{K} moiety having two functional groups capable of serving as initiation sites for peptide synthesis and a third functional group (e.g., a carboxyl group or an amino group) that enables binding to another molecular moiety (e.g., as may be present on the surface of a solid support). In this case, the two peptide monomers may be synthesized directly onto two reactive nitrogen groups of the linker L_{K} moiety in a variation of the solid phase synthesis technique. Such synthesis may be sequential or simultaneous.

Where sequential synthesis of the peptide chains of a dimer onto a linker is to be performed, two amine functional groups on the linker molecule are protected with two different orthogonally removable amine protecting groups. In preferred embodiments, the protected diamine is a protected lysine. The protected linker is coupled to a solid support via the linker's third functional group. The first amine protecting group is removed, and the first peptide of the dimer is synthesized on the first deprotected amine moiety. Then the second amine protecting group is removed, and the second peptide of the dimer is synthesized on the second deprotected amine moiety. For example, the first amino moiety of the linker may be protected with Alloc, and the second with Fmoc. In this case, the Fmoc group (but not the Alloc group) may be removed by treatment with a mild base [e.g., 20% piperidine in dimethyl formamide (DMF)], and the first peptide chain synthesized. Thereafter the Alloc group may be removed with a suitable reagent [e.g., Pd(PPh₃)/4-methyl morpholine and chloroform], and the second peptide chain synthesized. This technique may be used to generate dimers wherein the sequences of the two peptide chains are identical or different. Note that where different thiol-protecting groups for cysteine are to be used to control disulfide bond formation (as discussed below) this technique must be used even where the final amino acid sequences of the peptide chains of a dimer are identical.

Where simultaneous synthesis of the peptide chains of a dimer onto a linker is to be performed, two amine functional groups of the linker molecule are protected with the same removable amine protecting group. In preferred embodiments, the protected diamine is a protected lysine. The protected linker is coupled to a solid support via the linker's third functional group. In this case the two protected functional groups of the linker molecule are simultaneously deprotected, and the two peptide chains simultaneously synthesized on the deprotected amines. Note that using this technique, the sequences of the peptide chains of the dimer will be identical, and the thiol-protecting groups for the cysteine residues are all the same.

A preferred method for peptide synthesis is solid phase synthesis. Solid phase peptide synthesis procedures are well-known in the art [see, e.g., Stewart Solid Phase Peptide Syntheses (Freeman and Co.: San Francisco) 1969; 2002/2003 General Catalog from Novabiochem Corp, San Diego, USA; Goodman Synthesis of Peptides and Peptidomimetics (Houben-Weyl, Stuttgart) 2002]. In solid phase synthesis, synthesis is typically commenced from the C-terminal end of the peptide using an alpha.-amino protected resin. A suitable starting material can be prepared, for instance, by attaching the required .alpha.-amino acid to a chloromethylated resin, a hydroxymethyl resin, a polystyrene resin, a benzhydrylamine resin, or the like. One such chloromethylated resin is sold under the trade name BIO-BEADS SX-1 by Bio Rad Laboratories (Richmond, Calif.). The preparation of the hydroxymethyl resin has been described [Bodonszky, et al. (1966) Chem. Ind. London 38:1597]. The benzhydrylamine (BHA) resin has been described [Pietta and Marshall (1970) Chem. Commun. 650], and the hydrochloride form is commercially available from Beckman Instruments, Inc. (Palo Alto, Calif.). For example, an alpha.-amino protected amino acid may be coupled to a chloromethylated resin with the aid of a cesium bicarbonate catalyst, according to the method described by Gisin (1973) Helv. Chim. Acta 56:1467.

After initial coupling, the alpha-amino protecting group is removed, for example, using trifluoroacetic acid (TFA) or hydrochloric acid (HCl) solutions in organic solvents at room temperature. Thereafter, alpha-amino protected amino acids are successively coupled to a growing support-bound peptide chain. The alpha-amino protecting groups are those known to be useful in the art of stepwise synthesis of peptides, including: acyl-type protecting groups (e.g., formyl, trifluoroacetyl, acetyl), aromatic urethane-type protecting groups [e.g., benzyloxycarbonyl (Cbz) and substituted Cbz], aliphatic urethane protecting groups [e.g., t-butyloxycarbonyl (Boc), isopropyloxycarbonyl, cyclohexyloxycarbonyl], and alkyl type protecting groups (e.g., benzyl, triphenylmethyl), fluorenylmethyl oxycarbonyl (Fmoc), allyloxycarbonyl (Alloc), and 1-(4,4-dimethyl-2,6-dioxocyclohex-1-ylidene)ethyl (Dde).

The side chain protecting groups (typically ethers, esters, trityl, PMC (2,2,5,7,8-pentamethyl-chroman-6-sulphonyl), and the like) remain intact during coupling and is not split off during the deprotection of the amino-terminus protecting group or during coupling. The side chain protecting group must be removable upon the completion of the synthesis of the final peptide and under reaction conditions that will not alter the target peptide. The side chain protecting groups for Tyr include tetrahydropyranyl, tert-butyl, trityl, benzyl, Cbz, Z-Br—Cbz, and 2,5-dichlorobenzyl. The side chain protecting groups for Asp include benzyl, 2,6-dichlorobenzyl, methyl, ethyl, and cyclohexyl. The side chain protecting groups for Thr and Ser include acetyl, benzoyl, trityl, tetrahydropyranyl, benzyl, 2,6-dichlorobenzyl, and Cbz. The side chain protecting groups for Arg include nitro, Tosyl (Tos), Cbz, adamantyloxycarbonyl mesitylsulfonyl (Mts), 2,2,4,6,7-pentamethyldihydrobenzofurane-5-sulfonyl (Pbf), 4-methoxy-2,3,6-trimethyl-benzenesulfonyl (Mtr), or Boc. The side chain protecting groups for Lys include Cbz, 2-chlorobenzyloxycarbonyl (2-Cl—Cbz), 2-bromobenzyloxycarbonyl (2-Br—Cbz), Tos, or Boc.

After removal of the alpha-amino protecting group, the remaining protected amino acids are coupled stepwise in the desired order. Each protected amino acid is generally reacted in about a 3-fold excess using an appropriate carboxyl group activator such as 2-(1H-benzotriazol-1-yl)-1,1,3,3 tetramethyluronium hexafluorophosphate (HBTU) or dicyclohexylcarbodimide (DCC) in solution, for example, in methylene chloride (CH₂2Cl₂), N-methylpyrrolidone, dimethyl formamide (DMF), or mixtures thereof.

After the desired amino acid sequence has been completed, the desired peptide is decoupled from the resin support by treatment with a reagent, such as trifluoroacetic acid (TFA) or hydrogen fluoride (HF), which not only cleaves the peptide from the resin, but also cleaves all remaining side chain protecting groups. When a chloromethylated resin is used, hydrogen fluoride treatment results in the formation of the free peptide acids. When the benzhydrylamine resin is used, hydrogen fluoride treatment results directly in the free peptide amide. Alternatively, when the chloromethylated resin is employed, the side chain protected peptide can be decoupled by treatment of the peptide resin with ammonia to give the desired side chain protected amide or with an alkylamine to give a side chain protected alkylamide or dialkylamide. Side chain protection is then removed in the usual fashion by treatment with hydrogen fluoride to give the free amides, alkylamides, or dialkylamides. In preparing the esters of the invention, the resins used to prepare the peptide acids are employed, and the side chain protected peptide is cleaved with base and the appropriate alcohol (e.g., methanol). Side chain protecting groups are then removed in the usual fashion by treatment with hydrogen fluoride to obtain the desired ester.

These procedures can also be used to synthesize peptides in which amino acids other than the 20 naturally occurring, genetically encoded amino acids are substituted at one, two, or more positions of any of the compounds of the invention. Synthetic amino acids that can be substituted into the peptides of the present invention include, but are not limited to, N-methyl, L-hydroxypropyl, L-3,4-dihydroxyphenylalanyl, delta amino acids such as L-delta-hydroxylysyl and D-delta-methylalanyl, L-.alpha.-methylalanyl, .beta. amino acids, and isoquinolyl. D-amino acids and non-naturally occurring synthetic amino acids can also be incorporated into the peptides of the present invention.

### Peptide Modifications

One can also modify the amino and/or carboxy termini of the peptide compounds of the invention to produce other compounds of the invention. Amino terminus modifications include methylation (e.g., —NHCH₃ or —N(CH₃)₂), acetylation (e.g., with acetic acid or a halogenated derivative thereof such as .alpha.-chloroacetic acid, .alpha.-bromoacetic acid, or .alpha.-iodoacetic acid), adding a benzyloxycarbonyl (Cbz) group, or blocking the amino terminus with any blocking group containing a carboxylate functionality defined by RCOO— or sulfonyl functionality defined by R—SO₂—, where R is selected from alkyl, aryl, heteroaryl, alkyl aryl, and the like, and similar groups. One can also incorporate a desamino acid at the N-terminus (so that there is no N-terminal amino group) to decrease susceptibility to proteases or to restrict the conformation of the peptide compound. In preferred embodiments, the N-terminus is acetylated. In particularly preferred embodiments an N-terminal glycine is acetylated to yield N-acetylglycine (AcG).

Carboxy terminus modifications include replacing the free acid with a carboxamide group or forming a cyclic lactam at the carboxy terminus to introduce structural constraints. One can also cyclize the peptides of the invention, or incorporate a desamino or descarboxy residue at the termini of the peptide, so that there is no terminal amino or carboxyl group, to decrease susceptibility to proteases or to restrict the conformation of the peptide. C-terminal functional groups of the compounds of the present invention include amide, amide lower alkyl, amide di(lower alkyl), lower alkoxy, hydroxy, and carboxy, and the lower ester derivatives thereof, and the pharmaceutically acceptable salts thereof.

One can replace the naturally occurring side chains of the 20 genetically encoded amino acids (or the stereoisomeric D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclic. In particular, proline analogues in which the ring size of the proline residue is changed from 5 members to 4, 6, or 7 members can be employed. Cyclic groups can be saturated or unsaturated, and if unsaturated, can be aromatic or non-aromatic. Heterocyclic groups preferably contain one or more nitrogen, oxygen, and/or sulfur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl. These heterocyclic groups can be substituted or unsubstituted. Where a group is substituted, the substituent can be alkyl, alkoxy, halogen, oxygen, or substituted or unsubstituted phenyl.

One can also readily modify peptides by phosphorylation, and other methods [e.g., as described in Hruby, et al. (1990) Biochem J. 268:249-262]. The peptide compounds of the invention also serve as structural models for non-peptidic compounds with similar biological activity. Those of skill in the art recognize that a variety of techniques are available for constructing compounds with the same or similar desired biological activity as the lead peptide compound, but with more favorable activity than the lead with respect to solubility, stability, and susceptibility to hydrolysis and proteolysis [See, Morgan and Gainor (1989) Ann. Rep. Med. Chem. 24:243-252]. These techniques include replacing the peptide backbone with a backbone composed of phosphonates, amidates, carbamates, sulfonamides, secondary amines, and N-methylamino acids.

### Formation of Disulfide Bonds

The compounds of the present invention may contain one or more intramolecular disulfide bonds. In one embodiment, a peptide monomer or dimer comprises at least one intramolecular disulfide bond. In preferred embodiments, a peptide dimer comprises two intramolecular disulfide bonds.

Such disulfide bonds may be formed by oxidation of the cysteine residues of the peptide core sequence. In one embodiment the control of cysteine bond formation is exercised by choosing an oxidizing agent of the type and concentration effective to optimize formation of the desired isomer. For example, oxidation of a peptide dimer to form two intramolecular disulfide bonds (one on each peptide chain) is preferentially achieved (over formation of intermolecular disulfide bonds) when the oxidizing agent is DMSO.

In preferred embodiments, the formation of cysteine bonds is controlled by the selective use of thiol-protecting groups during peptide synthesis. For example, where a dimer with two intramolecular disulfide bonds is desired, the first monomer peptide chain is synthesized with the two cysteine residues of the core sequence protected with a first thiol protecting group [e.g., trityl(Trt), allyloxycarbonyl (Alloc), and 1-(4,4-dimethyl-2,6-dioxocyclohex-1-ylidene)ethyl (Dde) or the like], then the second monomer peptide is synthesized the two cysteine residues of the core sequence protected with a second thiol protecting group different from the first thiol protecting group [e.g., acetamidomethyl (Acm), t-butyl (tBu), or the like]. Thereafter, the first thiol protecting groups are removed effecting bisulfide cyclization of the first monomer, and then the second thiol protecting groups are removed effecting bisulfide cyclization of the second monomer.

Other embodiments of this invention provide for analogues of these disulfide derivatives in which one of the sulfurs has been replaced by a CH₂ group or other isotere for sulfur. These analogues can be prepared from the compounds of the present invention, wherein each core sequence contains at least one C or homocysteine residue and an α-amino-γ-butyric acid in place of the second C residue, via an intramolecular or intermolecular displacement, using methods known in the art [See, e.g., Barker, et al. (1992) J. Med. Chem. 35:2040-2048 and Or, et al. (1991) J. Org. Chem. 56:3146-3149]. One of skill in the art will readily appreciate that this displacement can also occur using other homologs of α-amino-γ-butyric acid and homocysteine.

In addition to the foregoing cyclization strategies, other non-disulfide peptide cyclization strategies can be employed. Such alternative cyclization strategies include, for example, amide-cyclization strategies as well as those involving the formation of thio-ether bonds. Thus, the compounds of the present invention can exist in a cyclized form with either an intramolecular amide bond or an intramolecular thio-ether bond. For example, a peptide may be synthesized wherein one cysteine of the core sequence is replaced with lysine and the second cysteine is replaced with glutamic acid. Thereafter a cyclic monomer may be formed through an amide bond between the side chains of these two residues. Alternatively, a peptide may be synthesized wherein one cysteine of the core sequence is replaced with lysine. A cyclic monomer may then be formed through a thio-ether linkage between the side chains of the lysine residue and the second cysteine residue of the core sequence. As such, in addition to disulfide cyclization strategies, amide-cyclization strategies and thio-ether cyclization strategies can both be readily used to cyclize the compounds of the present invention. Alternatively, the amino-terminus of the peptide can be capped with an .alpha.-substituted acetic acid, wherein the .alpha.-substituent is a leaving group, such as an .alpha.-haloacetic acid, for example, .alpha.-chloroacetic acid, .alpha.-bromoacetic acid, or .alpha.-iodoacetic acid.

### Addition of Linkers

In embodiments where a peptide dimer is dimerized by a linker L_{K} moiety, said linker may be incorporated into the peptide during peptide synthesis. For example, where a linker L_{K} moiety contains two functional groups capable of serving as initiation sites for peptide synthesis and a third functional group (e.g., a carboxyl group or an amino group) that enables binding to another molecular moiety, the linker may be conjugated to a solid support. Thereafter, two peptide monomers may be synthesized directly onto the two reactive nitrogen groups of the linker L_{K} moiety in a variation of the solid phase synthesis technique.

Scheme 1 is an example of a dimerization strategy for a peptide of the present invention.

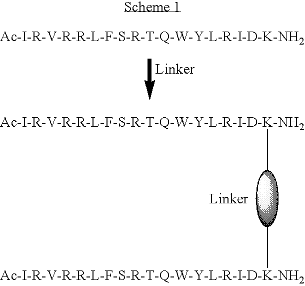

In alternate embodiments where a peptide dimer is dimerized by a linker L_{K} moiety, said linker may be conjugated to the two peptide monomers of a peptide dimer after peptide synthesis. Such conjugation may be achieved by methods well established in the art. In one embodiment, the linker contains at least two functional groups suitable for attachment to the target functional groups of the synthesized peptide monomers. For example, a linker with two free amine groups may be reacted with the C-terminal carboxyl groups of each of two peptide monomers. In another example, linkers containing two carboxyl groups, either preactivated or in the presence of a suitable coupling reagent, may be reacted with the N-terminal or side chain amine groups, or C-terminal lysine amides, of each of two peptide monomers.

### Addition of Spacers

In embodiments where the peptide compounds contain a spacer moiety, said spacer may be incorporated into the peptide during peptide synthesis. For example, where a spacer contains a free amino group and a second functional group (e.g., a carboxyl group or an amino group) that enables binding to another molecular moiety, the spacer may be conjugated to the solid support. Thereafter, the peptide may be synthesized directly onto the spacer's free amino group by standard solid phase techniques.

In a preferred embodiment, a spacer containing two functional groups is first coupled to the solid support via a first functional group. Next a linker L_{K} moiety having two functional groups capable of serving as initiation sites for peptide synthesis and a third functional group (e.g., a carboxyl group or an amino group) that enables binding to another molecular moiety is conjugated to the spacer via the spacer's second functional group and the linker's third functional group. Thereafter, two peptide monomers may be synthesized directly onto the two reactive nitrogen groups of the linker L_{K} moiety in a variation of the solid phase synthesis technique. For example, a solid support coupled spacer with a free amine group may be reacted with a lysine linker via the linker's free carboxyl group.

In alternate embodiments where the peptide compounds contain a spacer moiety, said spacer may be conjugated to the peptide after peptide synthesis. Such conjugation may be achieved by methods well established in the art. In one embodiment, the linker contains at least one functional group suitable for attachment to the target functional group of the synthesized peptide. For example, a spacer with a free amine group may be reacted with a peptide's C-terminal carboxyl group. In another example, a linker with a free carboxyl group may be reacted with the free amine group of a peptide's N-terminus or of a lysine residue. In yet another example, a spacer containing a free sulfhydryl group may be conjugated to a cysteine residue of a peptide by oxidation to form a disulfide bond.

### Attachment of Water Soluble Polymers

Included with the below description, the U.S. patent application Ser. No. 10/844,933 and International Patent Application No. PCT/US04/14887, filed May 12, 2004, are incorporated by reference herein in their entirety. In recent years, water-soluble polymers, such as polyethylene glycol (PEG), have been used for the covalent modification of peptides of therapeutic and diagnostic importance. Attachment of such polymers is thought to enhance biological activity, prolong blood circulation time, reduce immunogenicity, increase aqueous solubility, and enhance resistance to protease digestion. For example, covalent attachment of PEG to therapeutic polypeptides such as interleukins [Knauf, et al. (1988) J. Biol. Chem. 263; 15064; Tsutsumi, et al. (1995) J. Controlled Release 33:447), interferons (Kita, et al. (1990) Drug Des. Delivery 6:157), catalase (Abuchowski, et al. (1977) J. Biol. Chem. 252:582), superoxide dismutase (Beauchamp, et al. (1983) Anal. Biochem. 131:25), and adenosine deaminase (Chem, et al. (1981) Biochim. Biophy. Acta 660:293), has been reported to extend their half life in vivo, and/or reduce their immunogenicity and antigenicity.

The peptide compounds of the invention may further comprise one or more water soluble polymer moieties. Preferably, these polymers are covalently attached to the peptide compounds. The water soluble polymer may be, for example, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), poly(n-vinyl pyrrolidone)polyethylene glycol, propropylene glycol homopolymers, polypropylene oxide/ethylene oxide copolymers, and polyoxyethylated polyols. A preferred water soluble polymer is PEG.

Peptides, peptide dimers and other peptide-based molecules of the invention can be attached to water-soluble polymers (e.g., PEG) using any of a variety of chemistries to link the water-soluble polymer(s) to the receptor-binding portion of the molecule (e.g., peptide+spacer). A typical embodiment employs a single attachment junction for covalent attachment of the water soluble polymer(s) to the receptor-binding portion, however in alternative embodiments multiple attachment junctions may be used, including further variations wherein different species of water-soluble polymer are attached to the receptor-binding portion at distinct attachment junctions, which may include covalent attachment junction(s) to the spacer and/or to one or both peptide chains. In some embodiments, the dimer or higher order multimer will comprise distinct species of peptide chain (i.e., a heterodimer or other heteromultimer). By way of example and not limitation, a dimer may comprise a first peptide chain having a PEG attachment junction and the second peptide chain may either lack a PEG attachment junction or utilize a different linkage chemistry than the first peptide chain and in some variations the spacer may contain or lack a PEG attachment junction and said spacer, if PEGylated, may utilize a linkage chemistry different than that of the first and/or second peptide chains. An alternative embodiment employs a PEG attached to the spacer portion of the receptor-binding portion and a different water-soluble polymer (e.g., a carbohydrate) conjugated to a side chain of one of the amino acids of the peptide portion of the molecule.

A wide variety of polyethylene glycol (PEG) species may be used for PEGylation of the receptor-binding portion (peptides+spacer). Substantially any suitable reactive PEG reagent can be used. In preferred embodiments, the reactive PEG reagent will result in formation of a carbamate or amide bond upon conjugation to the receptor-binding portion. Suitable reactive PEG species include, but are not limited to, those which are available for sale in the Drug Delivery Systems catalog (2003) of NOF Corporation (Yebisu Garden Place Tower, 20-3 Ebisu 4-chome, Shibuya-ku, Tokyo 150-6019) and the Molecular Engineering catalog (2003) of Nektar Therapeutics (490 Discovery Drive, Huntsville, Ala. 35806). For example and not limitation, the following PEG reagents are often preferred in various embodiments: mPEG2-NHS, mPEG2-ALD, multi-Arm PEG, mPEG(MAL)2, mPEG2(MAL), mPEG-NH2, mPEG-SPA, mPEG-SBA, mPEG-thioesters, mPEG-Double Esters, mPEG-BTC, mPEG-ButyrALD, mPEG-ACET, heterofunctional PEGs (NH2-PEG-COOH, Boc-PEG-NHS, Fmoc-PEG-NHS, NHS-PEG-VS, NHS-PEG-MAL), PEG acrylates (ACRL-PEG-NHS), PEG-phospholipids (e.g., mPEG-DSPE), multiarmed PEGs of the SUNBRITE series including the GL series of glycerine-based PEGs activated by a chemistry chosen by those skilled in the art, any of the SUNBRITE activated PEGs (including but not limited to carboxyl-PEGs, p-NP-PEGs, Tresyl-PEGs, aldehyde PEGs, acetal-PEGs, amino-PEGs, thiol-PEGs, maleimido-PEGs, hydroxyl-PEG-amine, amino-PEG-COOH, hydroxyl-PEG-aldehyde, carboxylic anhydride type-PEG, functionalized PEG-phospholipid, and other similar and/or suitable reactive PEGs as selected by those skilled in the art for their particular application and usage.

The polymer may be of any molecular weight, and may be branched or unbranched. A preferred PEG for use in the present invention comprises linear, unbranched PEG having a molecular weight of from about 20 kilodaltons (kD or kDa) to about 40 kD (the term “about” indicating that in preparations of PEG, some molecules will weigh more, some less, than the stated molecular weight). Most preferably, the PEG has a molecular weight of from about 30 kD to about 40 kD. Other sizes may be used, depending on the desired therapeutic profile (e.g., duration of sustained release desired; effects, if any, on biological activity; ease in handling; degree or lack of antigenicity; and other known effects of PEG on a therapeutic peptide).

The number of polymer molecules attached may vary; for example, one, two, three, or more water soluble polymers may be attached to a KGF-R binding peptide of the invention. The multiple attached polymers may be the same or different chemical moieties (e.g., PEGs of different molecular weight). In some cases, the degree of polymer attachment (the number of polymer moieties attached to a peptide and/or the total number of peptides to which a polymer is attached) may be influenced by the proportion of polymer molecules versus peptide molecules in an attachment reaction, as well as by the total concentration of each in the reaction mixture. In general, the optimum polymer versus peptide ratio (in terms of reaction efficiency to provide for no excess unreacted peptides and/or polymer moieties) will be determined by factors such as the desired degree of polymer attachment (e.g., mono, di-, tri-, etc.), the molecular weight of the polymer selected, whether the polymer is branched or unbranched, and the reaction conditions for a particular attachment method.

In preferred embodiments, the covalently attached water soluble polymer is PEG. For illustrative purposes, examples of methods for covalent attachment of PEG (PEGylation) are described below. These illustrative descriptions are not intended to be limiting. One of ordinary skill in the art will appreciate that a variety of methods for covalent attachment of a broad range of water soluble polymers is well established in the art. As such, peptide compounds to which any of a number of water soluble polymers known in the art have been attached by any of a number of attachment methods known in the art are encompassed by the present invention.

In one embodiment, PEG may serve as a linker that dimerizes two peptide monomers. In one embodiment, PEG is attached to at least one terminus (N-terminus or C-terminus) of a peptide monomer or dimer. In another embodiment PEG is attached to a spacer moiety of a peptide monomer or dimer. In a preferred embodiment PEG is attached to the linker moiety of a peptide dimer. In a highly preferred embodiment, PEG is attached to a spacer moiety, where said spacer moiety is attached to the linker L.sub.K moiety that connects the monomers of a peptide dimer. Most preferably, PEG is attached to a spacer moiety, where said spacer moiety is attached to a peptide dimer via the carbonyl carbon of a lysine linker, or the amide nitrogen of a lysine amide linker.

There are a number of PEG attachment methods available to those skilled in the art [see, e.g., Goodson, et al. (1990) Bio/Technology 8:343 (PEGylation of interleukin-2 at its glycosylation site after site-directed mutagenesis); EP 0 401 384 (coupling PEG to G-CSF); Malik, et al., (1992) Exp. Hematol. 20:1028-1035 (PEGylation of GM-CSF using tresyl chloride); PCT Pub. No. WO 90/12874 (PEGylation of erythropoietin containing a recombinantly introduced cysteine residue using a cysteine-specific mPEG derivative); U.S. Pat. No. 5,757,078 (PEGylation of EPO peptides); and U.S. Pat. No. 6,077,939 (PEGylation of an N-terminal alpha.-carbon of a peptide)].

For example, PEG may be covalently bound to amino acid residues via a reactive group. Reactive groups are those to which an activated PEG molecule may be bound (e.g., a free amino or carboxyl group). For example, N-terminal amino acid residues and lysine (K) residues have a free amino group; and C-terminal amino acid residues have a free carboxyl group. Sulfhydryl groups (e.g., as found on cysteine residues) may also be used as a reactive group for attaching PEG. In addition, enzyme-assisted methods for introducing activated groups (e.g., hydrazide, aldehyde, and aromatic-amino groups) specifically at the C-terminus of a polypeptide have been described [Schwarz, et al. (1990) Methods Enzymol. 184:160; Rose, et al. (1991) Bioconjugate Chem. 2:154; Gaertner, et al. (1994) J. Biol. Chem. 269:7224].

For example, PEG molecules may be attached to peptide amino groups using methoxylated PEG (“mPEG”) having different reactive moieties. Such polymers include mPEG-succinimidyl succinate, mPEG-succinimidyl carbonate, mPEG-imidate, mPEG-4-nitrophenyl carbonate, and mPEG-cyanuric chloride. Similarly, PEG molecules may be attached to peptide carboxyl groups using methoxylated PEG with a free amine group (mPEG-NH₂).

Where attachment of the PEG is non-specific and a peptide containing a specific PEG attachment is desired, the desired PEGylated compound may be purified from the mixture of PEGylated compounds. For example, if an N-terminally PEGylated peptide is desired, the N-terminally PEGylated form may be purified from a population of randomly PEGylated peptides (i.e., separating this moiety from other monoPEGylated moieties).

In preferred embodiments, PEG is attached site-specifically to a peptide. Site-specific PEGylation at the N-terminus, side chain, and C-terminus of a potent analog of growth hormone-releasing factor has been performed through solid-phase synthesis [Felix, et al. (1995) Int. J. Peptide Protein Res. 46:253]. Another site-specific method involves attaching a peptide to extremities of liposomal surface-grafted PEG chains in a site-specific manner through a reactive aldehyde group at the N-terminus generated by sodium periodate oxidation of N-terminal threonine [Zalipsky, et al. (1995) Bioconj. Chem. 6:705]. However, this method is limited to polypeptides with N-terminal serine or threonine residues. Another site-specific method for N-terminal PEGylation of a peptide via a hydrazone, reduced hydrazone, oxime, or reduced oxime bond is described in U.S. Pat. No. 6,077,939 to Wei, et al.

In one method, selective N-terminal PEGylation may be accomplished by reductive alkylation which exploits differential reactivity of different types of primary amino groups (lysine versus the N-terminal) available for derivatization in a particular protein. Under the appropriate reaction conditions, a carbonyl group containing PEG is selective attached to the N-terminus of a peptide. For example, one may selectively N-terminally PEGylate the protein by performing the reaction at a pH which exploits the pK.sub.a differences between the C-amino groups of a lysine residue and the alpha.-amino group of the N-terminal residue of the peptide. By such selective attachment, PEGylation takes place predominantly at the N-terminus of the protein, with no significant modification of other reactive groups (e.g., lysine side chain amino groups). Using reductive alkylation, the PEG should have a single reactive aldehyde for coupling to the protein (e.g., PEG propionaldehyde may be used).

Site-specific mutagenesis is a further approach which may be used to prepare peptides for site-specific polymer attachment. By this method, the amino acid sequence of a peptide is designed to incorporate an appropriate reactive group at the desired position within the peptide. For example, WO 90/12874 describes the site-directed PEGylation of proteins modified by the insertion of cysteine residues or the substitution of other residues for cysteine residues. This publication also describes the preparation of mPEG-erythropoietin (“mPEG-EPO”) by reacting a cysteine-specific mPEG derivative with a recombinantly introduced cysteine residue on EPO.

Where PEG is attached to a spacer or linker moiety, similar attachment methods may be used. In this case, the linker or spacer contains a reactive group and an activated PEG molecule containing the appropriate complementary reactive group is used to effect covalent attachment. In preferred embodiments the linker or spacer reactive group contains a terminal amino group (i.e., positioned at the terminus of the linker or spacer) which is reacted with a suitably activated PEG molecule to make a stable covalent bond such as an amide or a carbamate. Suitable activated PEG species include, but are not limited to, mPEG.-para-nitrophenylcarbonate (mPEG-NPC), mPEG-succinimidyl carbonate (mPEG-SC), and mPEG-succinimidyl propionate (mPEG-SPA). In other preferred embodiments, the linker or spacer reactive group contains a carboxyl group capable of being activated to form a covalent bond with an amine-containing PEG molecule under suitable reaction conditions. Suitable PEG molecules include mPEG-NH₂ and suitable reaction conditions include carbodiimide-mediated amide formation or the like.

### KGF-R Binding Assays

The biological activity of the various peptide compounds of this invention (e.g., as KGF-R agonists) can be assayed by any of a variety of methods that are well known in the art. Non-limiting examples of certain, preferred assays are also described here. In vitro competitive binding assays quantitate the ability of a test peptide to compete with KGF for binding to KGF-R. For example, peptides derived from a native sequence of KGF may be tested in a competition binding assay for their ability to inhibit KGF binding of KGF-R. In such assays, the IC50 concentration is measured.

### Bioactivity Assays

The bioactivity of a KGF-R binding peptide monomer or peptide dimer of the present invention may be analyzed using certain techniques known in the art. For example, Balb/Mk cells may be used as described in Gospodarowicz et al. U.S. Pat. No. 7,265,089, incorporate herein by reference in its entirety. In particular, bioactivity can be assessed by the ability of a peptide to promote growth of Balb/Mk cells. Stock cultures of Balb/Mk cells can be grown and maintained in low calcium Dulbecco's modified Eagle medium (DMEM) supplemented with 10% fetal bovine serum, 0.25 ug/ml fungizone, and 10 ng/ml acidic FGF (aFGF). The cells can be incubated at 37° C. in a 10% CO₂ atmosphere with 99% humidity. For the bioactivity assay, the cells can be seeded in 12-well plates at a density of 5×10³ cells per well in 1 ml of medium as described above for the stock cultures, and as described in Gospodarowicz et al. J. Cell. Physiol. (1990) 142: 325-333. Ten microliter aliquots containing the desired peptide monomer or peptide dimer can be diluted into 1 ml of 0.2% (w/v) gelatin in phosphate buffered saline (PBS). Ten microliters of this dilution may be added to Balb/Mk cells seeded in 12-well cluster plates containing 22 mm wells, at 5×10³ cells per well, and a 10 ul aliquot of either the dilution or medium containing a 10 ng FGF positive control can be added to the cells every other day. After seven days in culture, the cells may be trypsinized and the final cell density can be determined using a Coulter™ counter (Coulter Electronics, Hialeah, Fla., USA). The cells are released from the plates by replacing the culture medium with a solution containing 0.9% NaCl, 0.01 M sodium phosphate (pH 7.4), 0.25% trypsin, and 0.02% EDTA (STV). The cells are incubated in this solution for 5-10 minutes at 37° C. and then the stock culture medium is added to the cells. The cells are then counted using a Coulter™ counter. The final cell density can be graphed as a function of peptide concentration. The peptide concentration may be graphed on a log scale. The activity of a peptide can be compared to the activity of a native form of KGF and the ability of a peptide of the present invention to stimulate KGF-R can be confirmed.

In addition, the bioactivity of a peptide monomer or peptide dimer of the present invention may be also analyzed using adrenal cortex-derived capillary endothelial cells (ACE) or adult bovine aortic endothelial cells (ABAE) cells. (see Gospodarowicz et al. U.S. Pat. No. 7,265,089, incorporate herein by reference in its entirety). A KGF-R binding peptide of the present invention may be characterized by its activity on vascular endothelial cells derived from large vessels (adult bovine aortic endothelial cells, ABAE) or capillary cells (adrenal cortex-derived capillary endothelial cells, ACE) as compared with a native form of FGF, such as basic FGF (bFGF) or acidic FGF (aFGF). To confirm this activity in the peptides, their biological activity on endothelial cells can be tested. Stock cultures of ABAE and ACE cells can be grown and maintained in Dulbecco's modified Eagle medium supplemented with 10% bovine serum, 0.25 ug/ml fungizone, and 2 ng/ml bFGF. The cells may be incubated at 37° C. with a 10% CO₂ concentration and 99% humidity. In such a mitogenic assay, either 5×10³ ABAE or ACE cells can be plated per well in 12-well plates in stock culture medium, as described in Gospodarowicz et. al. Proc. Natl. Acad. USA (1976) 73:4120-4124; Gospodarowicz et. al. J. Cell. Physiol. (1976) 127:121-136; and Gospodarowicz et. al. Proc. Natl. Acad. USA (1989) 86:7311-7315. Peptide monomers or peptide dimers at various saturating concentrations, as well as the native form of KGF may be added every other day. After 7 days in culture, cells can be trypsinized as described for the Balb/MK cell cultures above and the final cell density can be determined using a Coulter counter.

### Pharmaceutical Compositions

In yet another aspect of the present invention, pharmaceutical compositions of the above KGF-R binding peptides are provided. Conditions alleviated or modulated by the administration of such compositions include those indicated above. Such pharmaceutical compositions may be for administration by oral, parenteral (intramuscular, intraperitoneal, intravenous (IV) or subcutaneous injection), transdermal (either passively or using iontophoresis or electroporation), transmucosal (nasal, vaginal, rectal, or sublingual) routes of administration or using bioerodible inserts and can be formulated in dosage forms appropriate for each route of administration. In general, comprehended by the invention are pharmaceutical compositions comprising effective amounts of a KGF-R binding peptide, or derivative products, of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 20, Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder (e.g., lyophilized) form.

Oral Delivery. Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets, pellets, powders, or granules. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K. In: Modern Pharmaceutics Edited by G. S. Banker and C. T. Rhodes Chapter 10, 1979, herein incorporated by reference. In general, the formulation will include the KGF-R binding peptides (or chemically modified forms thereof) and inert ingredients which allow for protection against the stomach environment, and release of the biologically active material in the intestine.

Also contemplated for use herein are liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

The peptides may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. As discussed above, PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189].

For oral formulations, the location of release may be the stomach, the small intestine (the duodenum, the jejunem, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the peptide (or derivative) or by release of the peptide (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (i.e. powder), for liquid forms a soft gelatin shell may be used. The shell material of cachets could be thick starch or other edible paper. For pills, lozenges, molded tablets or tablet triturates, moist massing techniques can be used.

The peptide (or derivative) can be included in the formulation as fine multiparticulates in the form of granules or pellets of particle size about 1 mm. The formulation of the material for capsule administration could also be as a powder, lightly compressed plugs, or even as tablets. These therapeutics could be prepared by compression.

Colorants and/or flavoring agents may also be included. For example, the peptide (or derivative) may be formulated (such as by liposome or microsphere encapsulation) and then further contained within an edible product, such as a refrigerated beverage containing colorants and flavoring agents.

One may dilute or increase the volume of the peptide (or derivative) with an inert material. These diluents could include carbohydrates, especially mannitol, .alpha.-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. The disintegrants may also be insoluble cationic exchange resins. Powdered gums may be used as disintegrants and as binders and can include powdered gums such as agar, Karaya or tragacanth. Alginic acid and its sodium salt are also useful as disintegrants.

Binders may be used to hold the peptide (or derivative) agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the peptide (or derivative).

An antifrictional agent may be included in the formulation of the peptide (or derivative) to prevent sticking during the formulation process. Lubricants may be used as a layer between the peptide (or derivative) and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the peptide (or derivative) into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants could be present in the formulation of the protein or derivative either alone or as a mixture in different ratios.

Additives which potentially enhance uptake of the peptide (or derivative) are for instance the fatty acids oleic acid, linoleic acid and linolenic acid.

Controlled release oral formulations may be desirable. The peptide (or derivative) could be incorporated into an inert matrix which permits release by either diffusion or leaching mechanisms, e.g., gums. Slowly degenerating matrices may also be incorporated into the formulation. Some enteric coatings also have a delayed release effect. Another form of a controlled release is by a method based on the Oros therapeutic system (Alza Corp.), i.e. the drug is enclosed in a semipermeable membrane which allows water to enter and push drug out through a single small opening due to osmotic effects.

Other coatings may be used for the formulation. These include a variety of sugars which could be applied in a coating pan. The peptide (or derivative) could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan coater or in a fluidized bed or by compression coating.

Parenteral Delivery. Preparations according to this invention for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Rectal or Vaginal Delivery. Compositions for rectal or vaginal administration are preferably suppositories which may contain, in addition to the active substance, excipients such as cocoa butter or a suppository wax. Compositions for nasal or sublingual administration are also prepared with standard excipients well known in the art.

Pulmonary Delivery. Also contemplated herein is pulmonary delivery of the KGF-R binding peptides (or derivatives thereof). The peptide (or derivative) is delivered to the lungs of a mammal while inhaling and traverses across the lung epithelial lining to the blood stream [see, e.g., Adjei, et al. (1990) Pharmaceutical Research 7:565-569; Adjei, et al. (1990) Int. J. Pharmaceutics 63:135-144 (leuprolide acetate); Braquet, et al. (1989) J. Cardiovascular Pharmacology 13(sup5):143-146 (endothelin-1); Hubbard, et al. (1989) Annals of Internal Medicine, Vol. III, pp. 206-212 (.alpha.1-antitrypsin); Smith, et al. (1989) J. Clin. Invest. 84:1145-1146 (.alpha.-1-proteinase); Oswein, et al. (1990) “Aerosolization of Proteins”, Proceedings of Symposium on Respiratory Drug Delivery II Keystone, Colorado (recombinant human growth hormone); Debs, et al. (1988) J. Immunol. 140:3482-3488 (interferon-.gamma. and tumor necrosis factor alpha.); and U.S. Pat. No. 5,284,656 to Platz, et al. (granulocyte colony stimulating factor). A method and composition for pulmonary delivery of drugs for systemic effect is described in U.S. Pat. No. 5,451,569 to Wong, et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.).

All such devices require the use of formulations suitable for the dispensing of peptide (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified peptides may also be prepared in different formulations depending on the type of chemical modification or the type of device employed.

Formulations suitable for use with a nebulizer, either jet or ultrasonic, will typically comprise peptide (or derivative) dissolved in water at a concentration of about 0.1 to 25 mg of biologically active protein per mL of solution. The formulation may also include a buffer and a simple sugar (e.g., for protein stabilization and regulation of osmotic pressure). The nebulizer formulation may also contain a surfactant, to reduce or prevent surface induced aggregation of the peptide (or derivative) caused by atomization of the solution in forming the aerosol.

Formulations for use with a metered-dose inhaler deyice will generally comprise a finely divided powder containing the peptide (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing peptide (or derivative) and may also include a bulking agent, such as lactose, sorbitol, sucrose, or mannitol in amounts which facilitate dispersal of the powder from the device, e.g., 50 to 90% by weight of the formulation. The peptide (or derivative) should most advantageously be prepared in particulate form with an average particle size of less than 10 mm (or microns), most preferably 0.5 to 5 mm, for most effective delivery to the distal lung.

Nasal Delivery. Nasal delivery of the KGF-R binding peptides (or derivatives) is also contemplated. Nasal delivery allows the passage of the peptide to the blood stream directly after administering the therapeutic product to the nose, without the necessity for deposition of the product in the lung. Formulations for nasal delivery include those with dextran or cyclodextran.

Other penetration-enhancers used to facilitate nasal delivery are also contemplated for use with the peptides of the present invention (such as described in International Patent Publication No. WO 2004056314, filed Dec. 17, 2003, incorporated herein by reference in its entirety).

Dosages. For all of the peptide compounds, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally dosage levels of 0.001 to 10 mg/kg of body weight daily are administered to mammals. Generally, for intravenous injection or infusion, dosage may be lower.

### Uses of the Peptides

The peptides of the invention are useful in vitro as tools for understanding the biological role of KGF, including the evaluation of the many factors thought to influence, and be influenced by, the production of KGF and the binding of KGF to the KGF-R (e.g., the mechanism of KGF/KGF-R signal transduction/receptor activation). The present peptides are also useful in the development of other compounds that bind to the KGF-R, because the present compounds provide important structure-activity-relationship information that facilitate that development.

Moreover, based on their ability to bind to KGF-R, the peptides of the present invention can be used as reagents for detecting KGF-R on living cells; fixed cells; in biological fluids; in tissue homogenates; in purified, natural biological materials; etc. For example, by labeling such peptides, one can identify cells having KGF-R on their surfaces. In addition, based on their ability to bind KGF-R, the peptides of the present invention can be used in in situ staining, FACS (fluorescence-activated cell sorting) analysis, Western blotting, ELISA (enzyme-linked immunosorbent assay), etc. In addition, based on their ability to bind to KGF-R, the peptides of the present invention can be used in receptor purification, or in purifying cells expressing KGF-R on the cell surface (or inside permeabilized cells).

The peptides of the invention can also be utilized as commercial reagents for various medical research and diagnostic purposes. Such uses can include but are not limited to: (1) use as a calibration standard for quantitating the activities of candidate KGF-R agonists in a variety of functional assays; (2) use as blocking reagents in random peptide screening, i.e., in looking for new families of KGF-R peptide ligands, the peptides can be used to block recovery of KGF peptides of the present invention; (3) use in co-crystallization with KGF-R, i.e., crystals of the peptides of the present invention bound to the KGF-R may be formed, enabling determination of receptor/peptide structure by X-ray crystallography; and (4) other research and diagnostic applications wherein the KGF-R is preferably activated or such activation is conveniently calibrated against a known quantity of an KGF-R agonist, and the like.

In yet another aspect of the present invention, methods of treatment and manufacture of a medicament are provided. The peptide compounds of the invention may be administered to warm blooded animals, including humans, to simulate the binding of KGF to the KGF-R in vivo. Thus, the present invention encompasses methods for therapeutic treatment of a disorder characterized by a need for epithelial cell proliferation or a disorder associated with a deficiency of KGF, which methods comprise administering a peptide of the invention in amounts sufficient to stimulate the KGF-R and thus, alleviate the symptoms associated with such disorders in vivo. The peptides of the present invention will find use in the treatment of any disorder where the stimulation of epithelial cell proliferation is desirable. For example, the disorders include, without limitation, various types of wounds discussed herein, scarring, side effects associated with chemotherapy, oral mucositis, venous ulcers, diabetic ulcers, decubitus ulcers, gastrointestinal disorders (e.g. ulcerative colitis), and ophthalmic disorders. In one embodiment, the present invention provides a method of treatment including the step of administering to a subject with a disorder described herein a peptide monomer or dimer described herein, such that the monomer or dimer has one or more of the following effects (i) binding of KGF-R; (ii) functions as an agonist of KGF-R; and (iii) induces epithelial cell proliferation, in the subject to which the monomer or dimer is administered.

Further details of the invention are illustrated by the following non-limiting Example. The disclosures of all citations, including issued patents, published applications, and scientific articles, in the specification are expressly incorporated herein by reference in their entirety.

### EXAMPLE 1

### High-affinity FGFR2 Binding Peptides Derived from the Native Epitope of the KGF Ligand

A series of high affinity peptides based on the native sequence of the RTQ loop (residues 65-80) of KGF were synthesized and tested for their ability to inhibit the binding of KGF to its receptor, KGFR (FGFR2IIIb). The structures of those synthetic peptides were optimized primarily by varying their length at the N- and C-termini, mutating some of the key residues at the N- and C-termini, and introducing an intramolecular disulfide constraint with various loop sizes. In addition, the position of the native RTQ epitope was varied within the peptide sequence. The peptide monomers were dimerized via a lysine at their C-terminus using a small molecular bi-functional linker to give the corresponding peptide dimers. Both the monomers and dimers were tested for their ability to inhibit the KGF/KGFR interaction. The most potent peptide dimers inhibited KGF binding with an IC50 of 4˜8 nM, while the most potent peptide monomers inhibited KGF binding with an IC50 of 15˜97 nM. These results demonstrate that these native epitope derived synthetic peptides bind directly to the KGFR with high affinity and can effectively compete with KGF for the KGFR binding site.

Results and Discussion. Synthetic peptides (Table 2) were synthesized using Fmoc chemistry on TentaGel R RAM (0.18 mmol/g, 400 mg) resins in a PTI Symphony peptide synthesizer. The N-terminal NH₂-groups were capped with Ac₂O capping agent. Following deprotection and cleavage with 85% TFA, 10% TIS, 2.5% H₂O and 2.5% Thioanisole, the crude peptides were precipitated from ether, purified by preparative RP-HPLC using linear gradients of acetonitrile (containing 0.1% TFA) in H₂O (containing 0.15% TFA) on Waters RCM Delta-Pak (C18, 200 Å, 10 mm, 40×200 mm) columns and lyophilized.

TABLE 2

| | |
|---|---|
| En- | |
| try | Sequences |

| | |
|---|---|
| 1 | Ac I R V R R L F S R T Q W Y L R I D R R K NH₂ |
| 2 | Ac R V R R L F S R T Q W Y L R I D R R K NH₂ |
| 3 | Ac V R R L F S R T Q W Y L R I D R R K NH₂ |
| 4 | Ac R R L F S R T Q W Y L R I D R R K NH₂ |
| 5 | Ac R L F S R T Q W Y L R I D R R K NH₂ |
| 6 | Ac I R V R R L F S R T Q W Y L R I D R K NH₂ |
| 7 | Ac R V R R L F S R T Q W Y L R I D R K NH₂ |
| 8 | Ac V R R L F S R T Q W Y L R I D R K NH₂ |
| 9 | Ac R R L F S R T Q W Y L R I D R K NH₂ |
| 10 | Ac R L F S R T Q W Y L R I D R K NH₂ |
| 11 | Ac I R V R R L F S R T Q W Y L R I D K NH₂ |
| 12 | Ac R V R R L F S R T Q W Y L R I D K NH₂ |
| 13 | Ac V R R L F S R T Q W Y L R I D K NH₂ |
| 14 | Ac R R L F S R T Q W Y L R I D K NH₂ |
| 15 | Ac R L F S R T Q W Y L R I D K NH₂ |
| 16 | Ac I R V R R C F S R T Q W Y C R I D R K NH₂ |
| 17 | Ac R V R R C F S R T Q W Y C R I D R K NH₂ |
| 18 | Ac V R R C F S R T Q W Y C R I D R K NH₂ |
| 19 | Ac I R V R C L F S R T Q W Y C R I D R K NH₂ |
| 20 | Ac R V R C L F S R T Q W Y C R I D R K NH₂ |
| 21 | Ac R V R C L F S R T Q W Y C R I D R K NH₂ |
| 21 | Ac R V R C L F S R T Q W Y C R I D Q K NH₂ |
| 22 | Ac I R V R R C F S R T Q W Y L C I D R K NH₂ |
| 23 | Ac R V R R C F S R T Q W Y L C I D R K NH₂ |
| 24 | Ac V R R C F S R T Q W Y L C I D R K NH₂ |
| 25 | Ac I R V R C L F S R T Q W Y L C I D R K NH₂ |
| 26 | Ac R V R C L F S R T Q W Y L C I D R K NH₂ |
| 27 | Ac V R C L F S R T Q W Y L C I D R K NH₂ |

An initial peptide monomer was synthesized and tested in a competition binding assay, and was found to inhibit KGF binding with an IC50 of 629 nM. The peptide sequences were optimized by varying their length at the N- and C-termini (Table 1, entries 1-15), and the peptide monomers were dimerized via a lysine at their C-terminus via a bi-functional linker (Scheme 1). The lead peptide dimers inhibited KGF binding with an IC50 of 4˜8 nM, while the most potent peptide monomers inhibited binding with IC50 of 15˜97 nM. The peptide architectures were also modified by introducing a disulfide constraint with various loop sizes in their sequences (Table 1, entries 16-27), and the most potent peptide with the disulfide constraint inhibited binding with an IC50 of 8 nM.

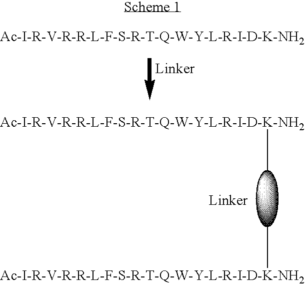

We claim:

1. A peptide comprising an amino acid sequence Xaa_{b}-Phe-Ser-Arg-Thr-Gln-Trp-Tyr-Xaa_{c} (SEQ ID NO:1) that binds KGF receptor, wherein Xaa_{b} is between 0 and 6 amino acids and Xaa_{c} is between 0 and 7 amino acids.

2. The peptide of claim 1 wherein Xaa_{b} is Ile-Arg-Val-Arg-Xaa₁-Xaa₂ (SEQ ID NO:2) and wherein Xaa₁ is Arg or Cys and Xaa₂ is Leu or Cys.

3. The peptide of claim 1 wherein Xaa_{b} is Arg-Val-Arg-Xaa₁-Xaa₂ and wherein Xaa₁ is Arg or Cys and Xaa₂ is Leu or Cys.

4. The peptide of claim 1 wherein Xaa_{b} is Val-Arg-Xaa₁-Xaa₂ and wherein Xaa₁ is Arg or Cys and Xaa₂ is Leu or Cys.

5. The peptide of claim 1 wherein Xaa_{b} is Arg-Xaa₁-Xaa₂ and wherein Xaa₁ is Arg or Cys and Xaa₂ is Leu or Cys.

6. The peptide of claim 1 wherein Xaa_{c} is Xaa₁-Xaa₂-Ile-Asp-Xaa₃-Xaa₄-Lys and wherein Xaa₁ is Leu or Cys; Xaa₂ is Arg or Cys; Xaa₃ is Arg, Lys, or Gln; and Xaa₄ is Arg or Lys.

7. The peptide of claim 1 wherein Xaa_{c} is Xaa₁-Xaa₂-Ile-Asp-Xaa₃-Xaa₄ and wherein Xaa₁ is Leu or Cys; Xaa₂ is Arg or Cys; Xaa₃ is Arg, Lys, or Gln; and Xaa₄ is Arg or Lys.

8. The peptide of claim 1 wherein Xaa_{c} is Xaa₁-Xaa₂-Ile-Asp-Xaa₃ and wherein Xaa₁ is Leu or Cys; Xaa₂ is Arg or Cys; and Xaa₃ is Arg, Lys, or Gln .

9. The peptide of claim 1 wherein the amino acid sequence is Phe-Ser-Arg-Thr-Gln-Trp-Tyr (SEQ ID NO:3).

10. The peptide of claim 1 wherein the N-terminus is acetylated.

11. The peptide of claim 1 wherein the amino acid sequence is a monomer.

12. The peptide of claim 1 wherein the amino acid sequence is a dimer.

13. The peptide of claim 1 wherein the amino acid sequence is a homodimer.

14. The peptide of claim 1 wherein the amino acid sequence is cyclized.

15. A peptide dimer, comprising: (a) a first peptide chain; (b) a second peptide chain; and (c) a linking moiety connecting said first and second peptide chains, wherein at least one of said peptide chains comprises the amino acid sequence of claim 1.

16. The peptide dimer of claim 15 wherein the amino acid sequence is Phe-Ser-Arg-Thr-Gln-Trp-Tyr (SEQ ID NO:3).

17. The peptide dimer of claim 15 wherein the linking moiety is a lysine residue.

18. A pharmaceutical composition comprising: (i) the peptide of claim 1; and (ii) a pharmaceutically acceptable carrier.

19. A pharmaceutical composition comprising: (i) the peptide dimer of claim 15; and (ii) a pharmaceutically acceptable carrier.

20. The pharmaceutical composition of claim 18 or 19 wherein the amino acid sequence is Phe-Ser-Arg-Thr-Gln-Trp-Tyr (SEQ ID NO:3).
